(19) United States
(12) Patent Application Publication
(10) Pub. No.: US 2026/0165631 A1
(43) Pub. Date: Jun. 18, 2026

(54) MEDICAL SYSTEM IMPLEMENTING A MACHINE LEARNING MODEL FOR DETECTION OF ATRIAL ARRHYTHMIA

(71) Applicant: Medtronic, Inc., Minneapolis, MN (US)

(72) Inventors: Shubha Majumder, Minneapolis, MN (US); Shantanu Sarkar, Roseville, MN (US); Sean R. Landman, Minneapolis, MN (US)

(21) Appl. No.: 19/124,999

(22) PCT Filed: Oct. 26, 2023
(86) PCT No.: PCT/US2023/077942

§ 371 Date: Apr. 28, 2025

Related U.S. Application Data
(60) Provisional application No. 63/381,518, filed on Oct. 28, 2022.

(51) Int. Cl. A61B 5/363; A61B 5/00; A61B 5/29; A61B 5/339; A61B 5/352; A61B 5/353; A61B 5/361

(52) U.S. Cl. CPC A61B 5/363; A61B 5/29; A61B 5/339; A61B 5/352; A61B 5/353; A61B 5/361; A61B 5/686

(57) ABSTRACT

A medical system comprises a medical device configured to store cardiac electrogram data of a patient from a predetermined, time period associated with an episode of atrial tachyarrhythmia of the patient detected by the medical device. The medical system further comprises processing circuitry configured to generate a set of features from the cardiac electrogram data, wherein, to generate at least one feature of the set of features, the processing circuitry′ is configured to modify the cardiac electrogram data to reduce QRS complexes in the cardiac electrogram data. The processing circuitry is further configured to determine a classification of the cardiac electrogram data from a plurality of predetermined classifications based on application of a machine learning model to the set of features, the plurality of classifications including at least one atrial tachycardia classification, and output an indication of the determined classification for presentation to a user via a computing device.

This application is an international application with provisional priority of U.S. Provisional Patent Application No. 63/381,518, filed 28 Oct. 2022, the entire contents of which are incorporated herein by reference.

### FIELD

The disclosure relates generally to medical systems and, more particularly, medical systems configured to monitor patient activity for changes in patient health.

### BACKGROUND

Some types of medical systems may monitor various patient data, e.g., cardiac electrogram (EGM) data, of a patient or a group of patients to detect changes in health. In some examples, the medical system may monitor the cardiac EGM data to detect one or more types of arrhythmia, such as bradycardia, tachycardia (e.g., atrial tachycardia), fibrillation (e.g., atrial fibrillation), or asystole (e.g., caused by sinus pause or AV block). In some examples, the medical system may include one or more of an implantable medical device or a wearable device to collect the data.

### SUMMARY

Medical systems and techniques as described herein include innovative application of machine learning models to a particular set features from cardiac EGM data to identify atrial tachycardia and other atrial tachyarrhythmias with significantly greater sensitivity and specificity than conventional approaches. The particular set of features focus the machine learning model on specific cardiac EGM data, e.g., data associated with atrial activity, that typify arrhythmia episodes of interest. In this manner, the techniques of this disclosure may improve the functioning of medical systems including medical devices that sense cardiac EGMs to help clinicians understand and protect the patient's cardiac health.

In some examples, processing circuitry of a medical system determines one or more features of the set of features based on a modified version of the cardiac EGM data. The modified version of the cardiac EGM data may reduce the presence of data associated with QRS complexes. The modification enhances these systems/techniques in a number of ways, particularly in terms of accuracy in detecting certain cardiac arrhythmias, such as atrial tachycardia (AT) and atrial fibrillation (AF). Conventional cardiac monitoring systems and techniques are configured to process the cardiac EGM data generally, which includes the more dominant ventricular activity in addition to atrial activity, often using rule-based analyses. Some features of the set of features may also utilize ventricular activity, but in the context of features designed to reveal atrial tachyarrhythmias. In this manner, the example medical systems may more accurately distinguish between a cardiac EGM depicting a true cardiac episode and one depicting no episode, resulting in more true detections (e.g., true positives) and/or fewer false detections (e.g., false positives and/or false negatives). In view of the above, the present disclosure describes a technological improvement or a technical solution that is integrated into a practical application.

Unlike conventional atrial arrhythmia detection systems, the techniques and systems of this disclosure may use a machine learning model to more accurately determine whether an atrial arrhythmia is present in cardiac EGM data collected by a medical device. In some examples, the machine learning model is trained with a set of training instances, where one or more of the training instances comprise data that indicate relationships between various EGM features (including features generated from a cardiac EGM modified to emphasize atrial activity and/or deemphasize ventricular activity as described herein) and classifications of different atrial arrhythmias. Because the machine learning model is trained with potentially thousands or millions of training instances, the machine learning model may reduce the amount of classification error in classifying cardiac EGM data as different atrial arrhythmia classifications when compared to conventional atrial arrhythmia detection systems.

Additionally, the techniques and systems of this disclosure may be implemented in an implantable medical device (IMD) that can continuously (e.g., on a periodic or triggered basis without human intervention) sense the cardiac EGM while subcutaneously implanted in a patient over months or years and perform numerous operations per second on patient EGM data to enable the systems herein to detect atrial arrhythmias. Using techniques of this disclosure with an IMD may be advantageous when a physician cannot be continuously present with the patient over weeks or months to evaluate the cardiac EGM and/or where performing the operations on the cardiac EGM described herein (e.g., modification of the cardiac EGM, generation of feature sets, and application of a machine learning model) on weeks or months of EGM data could not practically be performed in the mind of a physician.

Reducing classification errors for atrial arrhythmias with a machine learning model implementing techniques (e.g., modification of the cardiac EGM data and generation of features from the modified and unmodified cardiac EGM data) of this disclosure may provide one or more technical and clinical advantages. In some examples, a medical system modifying the cardiac EGM data to emphasize atrial components and/or deemphasize ventricular components and applying a trained machine learning model to resulting features may help the determination that cardiac EGM data of a particular episode has a particular atrial arrhythmia classification, e.g., with higher specificity and sensitivity. For example, when a system determines that a particular episode is a particular classification of atrial arrhythmia with higher specificity and sensitivity, a number of false positives may be reduced. This higher specificity and sensitivity may increase reliability of another device, user, and/or clinician on the accuracy of determining a patient's condition and improve resulting treatment of the patient and patient outcomes. Furthermore, accurately classifying atrial arrhythmia episodes, including reducing the number of false positives or other false classifications, when using techniques of this disclosure may decrease the clinical burden on physicians and other caregivers to review and identify episodes of arrhythmia.

In one example, a medical system comprises a medical device configured to store cardiac electrogram data of a patient from a predetermined time period associated with an episode of atrial tachyarrhythmia of the patient detected by the medical device, and processing circuitry. The processing circuitry is configured to generate a set of features from the cardiac electrogram data, wherein, to generate at least one feature of the set of features, the processing circuitry is configured to modify the cardiac electrogram data to reduce QRS complexes in the cardiac electrogram data, determine a classification of the cardiac electrogram data from a plurality of predetermined classifications based on application of a machine learning model to the set of features, the plurality of classifications including at least one atrial tachycardia classification, and output an indication of the determined classification for presentation to a user via a computing device.

In another example, a medical system comprises an insertable cardiac monitor. The insertable cardiac monitor comprises a housing configured for subcutaneous implantation in a patient, the housing having a length between 40 millimeters (mm) and 60 mm between a first end and a second end, a width less than the length, and a depth less than the width. The insertable cardiac monitor further comprises a first electrode at or proximate to the first end, a second electrode at or proximate to the second end, a memory within the housing, and first processing circuitry within the housing. The first processing circuitry is configured to detect an episode of atrial tachyarrhythmia of the patient based on a cardiac electrogram signal sensed via the first electrode and the second electrode, and store cardiac electrogram data from a predetermined time period associated with the episode in the memory based on the cardiac electrogram signal. The medical system further comprises one or more computing devices in communication with the insertable cardiac monitor. The one or more computing devices comprise second processing circuitry configured to generate a set of features from the cardiac electrogram data, wherein, to generate at least one feature of the set of features, the processing circuitry is configured to modify the cardiac electrogram data to reduce QRS complexes in the cardiac electrogram data, generate a two-dimensional array of samples comprising an arrangement of the set of features as respective sub-arrays of the two-dimensional array of samples, apply the two-dimensional array of samples as input to a machine learning model to determine a classification of the cardiac electrogram data from a plurality of predetermined classifications, the plurality of classifications including at least one atrial tachycardia classification, and output an indication of the determined classification for presentation to a user,

In another example, a method comprises generating, by processing circuitry, a set of features from cardiac electrogram data of a patient, wherein generating at least one feature of the set of features comprises modifying the cardiac electrogram data to reduce QRS complexes in the cardiac electrogram data, wherein the cardiac electrogram data was stored by a medical device and is from a predetermined time period associated with an episode of atrial tachyarrhythmia of the patient detected by a medical device. The method further comprises determining, by processing circuitry, a classification of the cardiac electrogram data from a plurality of predetermined classifications based on application of a machine learning model to the set of features, the plurality of classifications including at least one atrial tachycardia classification, and outputting, by processing circuitry, an indication of the determined classification for presentation to a user via a computing device.

In another example, a non-transitory computer-readable storage medium comprises program instructions that, when executed by processing circuitry of a medical system, cause the processing circuitry to perform any method described herein.

In another example, a medical system comprising a medical device configured to store cardiac electrogram data of a patient from a predetermined time period associated with an episode of atrial tachyarrhythmia of the patient detected by the medical device, The medical system further comprises means for performing any method described herein.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2A is a perspective drawing illustrating an insertable cardiac monitor.

FIG. 2B is a perspective drawing illustrating another insertable cardiac monitor.

FIG. 3 is a functional block diagram illustrating an example configuration of the medical device of FIG. 1, in accordance with one or more examples of the present disclosure.

FIG. 4 is a functional block diagram illustrating an example configuration of the external device of FIG. 1, in accordance with one or more examples of the present disclosure.

FIG. 5 is a block diagram illustrating an example system that includes a network and computing devices, which may be coupled to the medical device and external device of FIGS. 1-4, in accordance with one or more examples of the present disclosure.

FIG. 6 is a block diagram illustrating a logical perspective of an example service operated by the example system of FIG. 5, in accordance with one or more examples of the present disclosure.

FIG. 7A is a flow diagram illustrating an example operation for determining a classification of an atrial tachyarrhythmia episode, in accordance with one or more examples of the present disclosure,

FIG. 7B is a flow diagram illustrating another example operation for determining a classification of an atrial tachyarrhythmia episode, in accordance with one or more examples of the present disclosure.

FIGS. 8A and 8B illustrate example machine learning models, in accordance with one or more examples of the present disclosure.

FIGS. 9A and 9B depict graphical representations of cardiac EGM data collected by a medical device and other data derived from the cardiac EGM data.

FIG. 10A illustrates an example of input data for the example machine learning models of FIGS. 8A and 8B, in accordance with one or more examples of the present disclosure.

FIG. 10B illustrates an example of input data for the example machine learning models of FIGS. 8A and 8B, in accordance with one or more examples of the present disclosure.

FIG. 11 is a conceptual diagram illustrating an example training process for an machine learning model, in accordance with examples of the current disclosure.

Like reference characters denote like elements throughout the description and figures.

### DETAILED DESCRIPTION

In general, medical systems according to this disclosure implement techniques for determining a classification of cardiac electrogram (EGM) data associated with a cardiac episode in a patient. An example medical system includes at least one medical device or other sensor device (hereinafter referred to as a medical device) that is configured to detect cardiac episodes based on a cardiac EGM signal sensed using electrodes, and processing circuitry for executing an example technique for determining a classification of cardiac EGM data collected by the medical device for the episode. A variety of medical devices (e.g., implantable devices, wearable devices, etc.) may be configured to monitor cardiac EGM signals, e.g., electrocardiogram (ECG) signals, from electrodes, detect the cardiac episode by way of an application of a detection mechanism/method to sensed EGM signal, and store the associated cardiac EGM data.

The medical device may itself implement the techniques of this disclosure to determine the classification of the cardiac EGM data. In some examples, the medical device may transmit the cardiac EGM data for the episode to a computing device or cloud computing system for performing an application of the techniques for determining a classification of the cardiac EGM data, e.g., as part of an adjudication of the initial cardiac episode detection by the medical device. Example medical devices that may detect arrhythmia episodes and collect cardiac EGM data associated with the episodes may include an implantable or wearable monitoring device, such as the Reveal LINQ™ or LINQ II™ Insertable Cardiac Monitor (ICM), available from Medtronic, Inc. of Minneapolis, MN, a pacemaker/defibrillator, or a smartwatch, fitness tracking device, or other wearable sensor device.

Some of the techniques described herein improve the performance of medical systems classifying atrial tachyarrhythmias by modifying the cardiac EGM data to diminish non-atrial cardiac activity data (e.g., ventricular activity data). Such modifications may effectively reduce the influence of ventricular activity data on the classification, and focus the classification the atrial activity data to find indicia of one or more classifications of interest relating to atrial tachyarrhythmia. Implementing such improvements, the medical systems and techniques described herein realize an increase in accuracy for detecting true episodes, for instance, by detecting fewer false positives and overlooking fewer false negatives.

Some medical systems and techniques described herein leverage machine learning, for example, by employing a machine learning model to classify cardiac EGM data. In some examples, by introducing one or more features that are well-suited for representing the atrial activity of the patient, the machine learning model is more capable of distinguishing, for example, atrial tachycardia (AT) episodes from non-AT episodes and avoiding detection of false AT episodes. The present disclosure describes how such a model achieves a considerable level of accuracy (e.g., in terms of sensitivity and specificity).

Another example technique may be configured to improve upon the medical system's AT episode detection accuracy by calibrating the detection mechanism/method to the atrial activity of a particular patient (or patient group). Over time, the above-mentioned machine learning model may be further trained, for instance, specifically on a patient's device history. Hence, the techniques described in the present disclosure may personalize the patient's medical device to detect AT episodes more accurately in that patient. The techniques described herein may also (periodically) update the machine learning model of any given medical device, improving upon that medical device's AT detection logic with enhanced (e.g., more accurate) functionality. In some instances, that improvement may open a new application for legacy medical devices by rendering the legacy medical device's AT detection functionality more clinically beneficial.

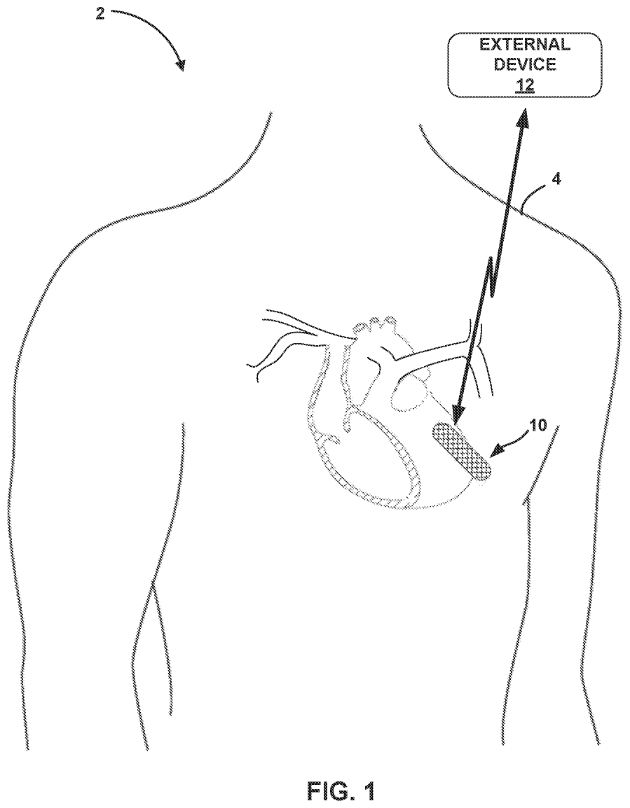

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 may be positioned on other locations, such as patient 4's cranium region. IMD 10 includes a plurality of electrodes (not shown in FIG. 1) and is configured to sense a cardiac EGM via the plurality of electrodes. In some examples, IMD 10 takes the form of the Reveal LINQ™ or LINQ TI™ ICM. In some examples, IMD 10 includes additional sensors, such as one or more sensors configured to sense patient activity, e.g., one or more accelerometers.

External device 12 may be a computing device with a display viewable by the user and an interface for receiving user input to external device 12. In some examples, external device 12 may be a notebook computer, tablet computer, workstation, one or more servers, cellular phone, personal digital assistant, or another computing device that may run an application that enables the computing device to interact with IMD 10. External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

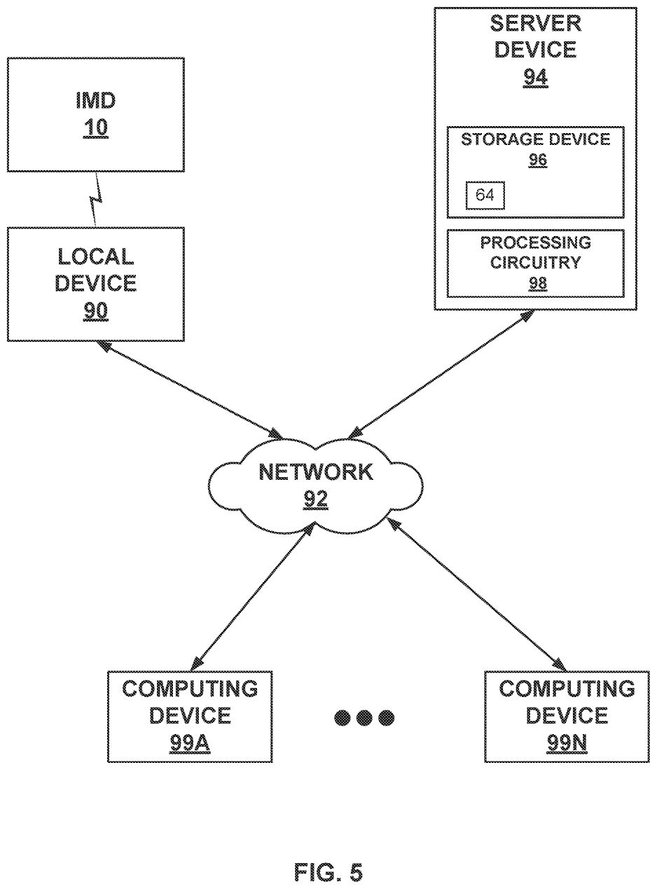

External device 12 may be used to configure operational parameters and/or device settings for IMD 10. External device 12 may be used to retrieve data from IMD 10. The retrieved data may include values of physiological parameters measured by IMD 10, indications of cardiac episodes (e.g., episodes of an arrhythmia) or other maladies detected by IMD 10, and physiological signals recorded by IMD 10. External device 12 may retrieve cardiac EGM segments recorded by IMD 10 due to IMD 10 determining that an episode of AT, AF, other atrial tachyarrhythmia, or another malady occurred during the segment, for example, as part of an adjudication operation. The cardiac EGM segments may comprise cardiac EGM data from a predetermined time period associated with the episode. As will be discussed in greater detail below with respect to FIG. 5, one or more remote computing devices may interact with IMD 10 in a manner similar to external device 12, e.g., to program IMD 10 and/or retrieve data from IMD 10, via a network.

Processing circuitry of medical system 2, e.g., of IMD 10, external device 12, and/or of one or more other computing devices, may be configured to perform the example techniques for classifying cardiac EGM data of an atrial tachyarrhythmia episode detected by IMD, e.g., as AT or other classifications, of this disclosure. The processing circuitry may identify certain waveforms (e.g., P-waves and QRS complexes) in cardiac EGM data, and then modify a portion of the cardiac EGM data to more accurately detect indicia of various classifications related to AT or other atrial arrhythmias. The processing circuitry may apply one or more machine learning models to a set of features determined from the cardiac EGM data and the modified cardiac EGM data to determine a classification of the cardiac EGM data.

Although described in the context of examples in which IMD 10 that senses patient cardiac activity may comprise an ICM, example systems including one or more implantable, wearable, or external devices of any type configured to sense cardiac EGMs may be configured to implement the techniques of this disclosure.

In some examples, a wearable device or other device may perform some or all of the techniques described herein in the same manner described herein with respect to IMD 10. In some examples, the wearable device operates with IMD 10 and/or external device 12 as potential providers of computing/storage resources and sensors for monitoring patient activity and other patient parameters. For example, the wearable device may communicate the patient's cardiac EGM data to external device 12 for storage in non-volatile memory and for applying a machine learning model to cardiac EGM data.

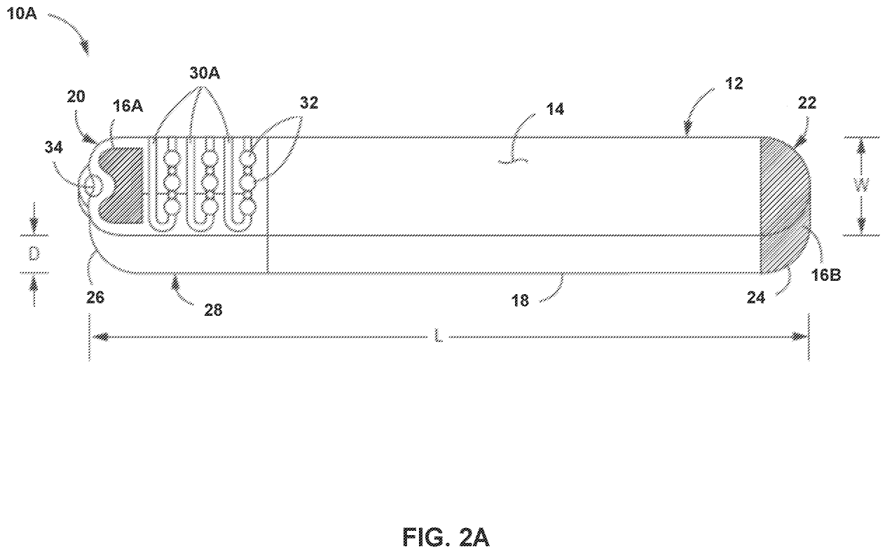

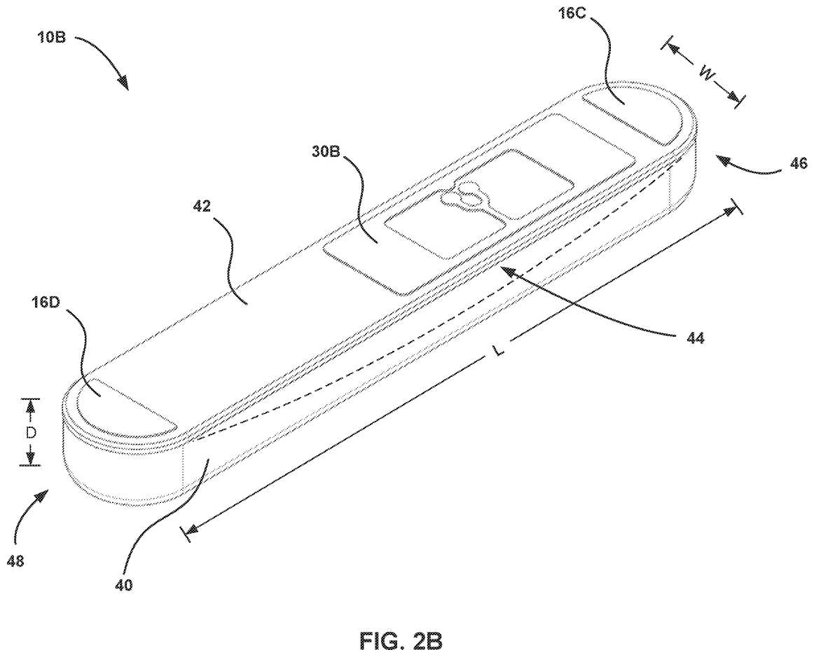

FIG. 2A is a perspective drawing illustrating an IMD 10A, which may be an example configuration of IMD 10 of FIG. 1 as an ICM. In the example shown in FIG. 2A, IMD 10A may be embodied as a monitoring device having housing 12, proximal electrode 16A and distal electrode 16B. Housing 12 may further comprise first major surface 14, second major surface 18, proximal end 20, and distal end 22. Housing 12 encloses electronic circuitry located inside the IMD 10A and protects the circuitry contained therein from body fluids. Housing 12 may be hermetically sealed and configured for subcutaneous implantation. Electrical feedthroughs provide electrical connection of electrodes 16A and 16B.

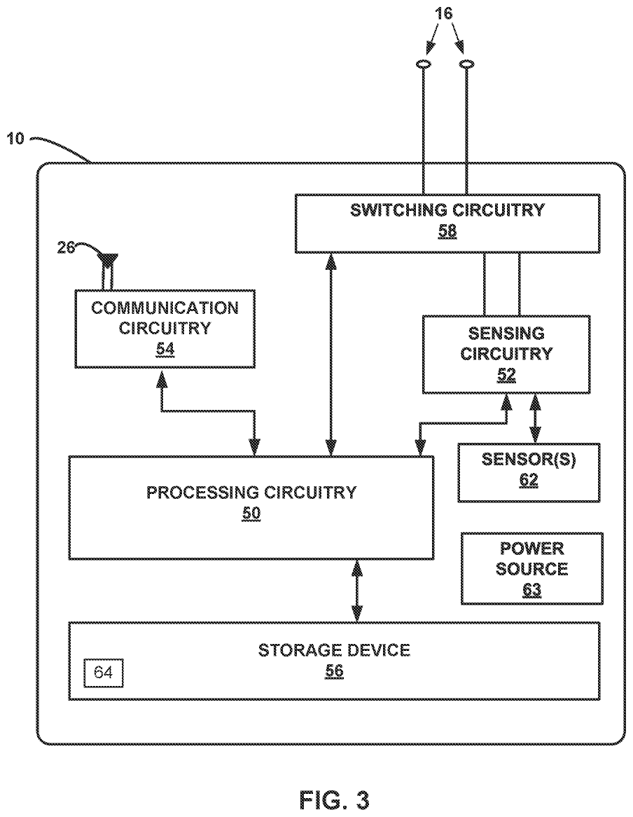

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D-is selected to allow IMD 10A to be inserted under the skin of the patient using a minimally invasive procedure and to remain in the desired orientation during insertion. For example, the device shown in FIG. 3A includes radial asymmetries (notably, the rectangular shape) along the longitudinal axis that maintains the device in the proper orientation following insertion. For example, the spacing between proximal electrode 46A and distal electrode 46B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within the patient, the first major surface 14 faces outward, toward the skin of the patient while the second major surface 18 is located opposite the first major surface 14. In addition, in the example shown in FIG. 2A, proximal end 20 and distal end 22 are rounded to reduce discomfort and irritation to surrounding tissue once inserted under the skin of the patient. IMD 10A, including instrument and method for inserting IMD 10 is described, for example, in U.S. Patent Publication No. 2014/0276928, incorporated herein by reference in its entirety.

Proximal electrode 16A is at or proximate to proximal end 20, and distal electrode 16B is at or proximate to distal end 22. Proximal electrode 16A and distal electrode 16B are used to sense cardiac EGM signals, e.g., ECG signals, thoracically outside the ribcage, which may be sub-muscularly or subcutaneously. EGM signals may be stored in a memory of IMD 10A, and data may be transmitted via integrated antenna 30A to another device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, or for measuring impedance, from any implanted location.

In the example shown in FIG. 2A, proximal electrode 16A is at or in close proximity to the proximal end 20 and distal electrode 16B is at or in close proximity to distal end 22. In this example, distal electrode 16B is not limited to a flattened, outward facing surface, but may extend from first major surface 14 around rounded edges 24 and/or end surface 26 and onto the second major surface 18 so that the electrode 16B has a three-dimensional curved configuration. In some examples, electrode 16B is an uninsulated portion of a metallic, e.g., titanium, part of housing 12.

In the example shown in FIG. 2A, proximal electrode 16A is located on first major surface 14 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 14 similar to that shown with respect to proximal electrode 16A.

The various electrode configurations allow for configurations in which proximal electrode 16A and distal electrode 16B are located on both first major surface 14 and second major surface 18. In other configurations, such as that shown in FIG. 2A, only one of proximal electrode 16A and distal electrode 16B is located on both major surfaces 14 and 18, and in still other configurations both proximal electrode 16A and distal electrode 16B are located on one of the first major surface 14 or the second major surface 18 (e.g., proximal electrode 16A located on first major surface 14 while distal electrode 16B is located on second major surface 18). In another example, IMD 10A may include electrodes on both major surface 14 and 18 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 2A, proximal end 20 includes a header assembly 28 that includes one or more of proximal electrode 16A, integrated antenna 30A, anti-migration projections 32, and/or suture hole 34. Integrated antenna 30A is located on the same major surface (i.e., first major surface 14) as proximal electrode 16A and is also included as part of header assembly 28. Integrated antenna 30A allows IMD 10A to transmit and/or receive data. In other examples, integrated antenna 30A may be formed on the opposite major surface as proximal electrode 16A, or may be incorporated within the housing 12 of IMD 10A. In the example shown in FIG. 2A, anti-migration projections 32 are located adjacent to integrated antenna 30A and protrude away from first major surface 14 to prevent longitudinal movement of the device. In the example shown in FIG. 2A, anti-migration projections 32 include a plurality (e.g., nine) small bumps or protrusions extending away from first major surface 14. As discussed above, in other examples anti-migration projections 32 may be located on the opposite major surface as proximal electrode 16A and/or integrated antenna 30A. In addition, in the example shown in FIG. 2A, header assembly 28 includes suture hole 34, which provides another means of securing IMD 10A to the patient to prevent movement following insertion. In the example shown, suture hole 34 is located adjacent to proximal electrode 16A. In one example, header assembly 28 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1 as an ICM. IMD 10B of FIG. 2B may be configured substantially similarly to IMD 10A of FIG. 2A, with differences between them discussed herein.

IMD 10B may include a leadless, subcutaneously-implantable monitoring device, e.g. an ICM. IMD 10B includes housing having a base 40 and an insulative cover 42. Proximal electrode 16C and distal electrode 16D may be formed or placed on an outer surface of cover 42. Various circuitries and components of IMD 10B, e.g., described below with respect to FIG. 3, may be formed or placed on an inner surface of cover 42, or within base 40. In some examples, a battery or other power source of IMD 10B may be included within base 40. In the illustrated example, antenna 30B is formed or placed on the outer surface of cover 42, but may be formed or placed on the inner surface in some examples. In some examples, insulative cover 42 may be positioned over an open base 40 such that base 40 and cover 42 enclose the circuitries and other components and protect them from fluids such as body fluids. The housing including base 70 and insulative cover 72 may be hermetically sealed and configured for subcutaneous implantation.

Circuitries and components may be formed on the inner side of insulative cover 42, such as by using flip-chip technology. Insulative cover 42 may be flipped onto a base 40. When flipped and placed onto base 40, the components of IMD 10B formed on the inner side of insulative cover 42 may be positioned in a gap 44 defined by base 40. Electrodes 16C and 16D and antenna 30B may be electrically connected to circuitry formed on the inner side of insulative cover 42 through one or more vias (not shown) formed through insulative cover 42. Insulative cover 42 may be formed of sapphire (i.e., corundum), glass, parylene, and/or any other suitable insulating material. Base 40 may be formed from titanium or any other suitable material (e.g., a biocompatible material).

Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 46C and distal electrode 46D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length Z may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within the patient, outer surface of cover 42 faces outward, toward the skin of the patient. In addition, as shown in FIG. 2B, proximal end 46 and distal end 48 are rounded to reduce discomfort and irritation to surrounding tissue once inserted under the skin of the patient. In addition, edges of IMD 10B may be rounded.

FIG. 3 is a functional block diagram illustrating an example configuration of IMD 10 of FIG. 1 in accordance with one or more techniques described herein. In the illustrated example, IMD 10 includes electrodes 16 (e.g., corresponding to any of electrodes 16A-16D), antenna 26, processing circuitry 50, sensing circuitry 52, communication circuitry 54, storage device 56, switching circuitry 58, and sensors 62. Processing circuitry 50 may be operatively coupled to sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples. IMD 10 further comprises a power source 63 to provide operational power for processing circuitry 50, sensing circuitry 52, communication circuitry 54, storage device 56, switching circuitry 58, and sensors 62.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to sense electrical signals of the heart of patient 4, for example by selecting the electrodes 16 and polarity, referred to as the sensing vector, used to sense a cardiac EGM, e.g., ECG, as controlled by processing circuitry 50. Sensing circuitry 52 may sense the cardiac EGM from electrodes 16 in order to facilitate monitoring the electrical activity of the heart. In some examples, sensing circuitry 52 may include one or more filters and amplifiers for filtering and amplifying signals received from electrodes 16 and/or sensors 62. Sensing circuitry 52 and processing circuitry 50 may store cardiac EGM data in storage device 56, e.g., of cardiac depolarizations/contractions or digitized samples of the electrical signals, for use by detection logic 64. Sensing circuitry 52 may also monitor signals from sensors 62, which may include one or more accelerometers, pressure sensors, and/or optical sensors, as examples. Sensing circuitry 52 may capture sensor signals from any one of sensors 62, e.g., to produce other patient data, in order to facilitate monitoring of patient activity and detecting changes in patient health.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include various patient data (e.g., patient physiological parameters such that those described herein corresponding to cardiac activity and episode data for detected atrial arrythmias).

IMD 10 is a medical device in which logic 64 implements an innovative detection method/mechanism (e.g., a machine learning model) that is well-suited to analyzing atrial activity in a patient's heart and in particular, detecting atrial arrythmia episodes. As described in greater detail below, other devise or systems, such as external device 12 or server device 94 (FIG. 5), e.g., of a cloud computing system, may additionally or alternatively implement logic 64 and the techniques described herein.

In accordance with the innovative detection method/mechanism, logic 64 may direct processing circuitry 50 to modify one or more portions of cardiac EGM data (e.g., waveforms) to reduce the presence of non-atrial activity, for example, by reducing their values (e.g., amplitude values). One purpose behind such a modification may be to diminish the influence of the non-atrial activity during application of the machine learning model; therefore, applying the machine learning model to the modified cardiac activity data focuses that model's computations on atrial activity. By modifying the non-atrial activity (and, in some examples, leaving any portion of atrial activity data unchanged), the innovative detection method/mechanism improves IMD 10's performance in terms of accuracy.

In some examples, processing circuitry 50 implements one or more non-machine learning techniques for initially detecting episodes of atrial tachyarrhythmia based on the sensed cardiac EGM or stored cardiac EGM data. An example technique for detecting atrial tachycardia is described in commonly-assigned U.S. patent application Ser. No. 17/339,308, titled “DETECTION OF ATRIAL TACHYCARDIA BASED ON REGULARITY OF CARDIAC RHYTHM,” which is incorporated herein in its entirety. Techniques for detecting atrial fibrillation may include criteria related to rate and regularity of R-waves in the cardiac EGM signal.

Based on detection of an episode atrial tachyarrhythmia using such techniques, processing circuitry 50 may store cardiac EGM data associated with the episode in memory 56 as episode data. Processing circuitry 50 may utilize logic 64 to implement the techniques of this disclosure to further process cardiac EGM data for the episode to determine a classification of the cardiac EGM data, e.g., as a true episode of an AT or atrial tachyarrhythmia of interest, or not a true episode. In some examples, processing circuitry 50 may control communication circuitry 54 to transmit the episode data to another device, e.g., external device 12 or a cloud computing system comprising one or more computing devices, for analysis according to the techniques of this disclosure, In this manner, the techniques of this disclosure may advantageously enable improved accuracy in the detection of changes in patient health and, consequently, better evaluation of the condition of the patient.

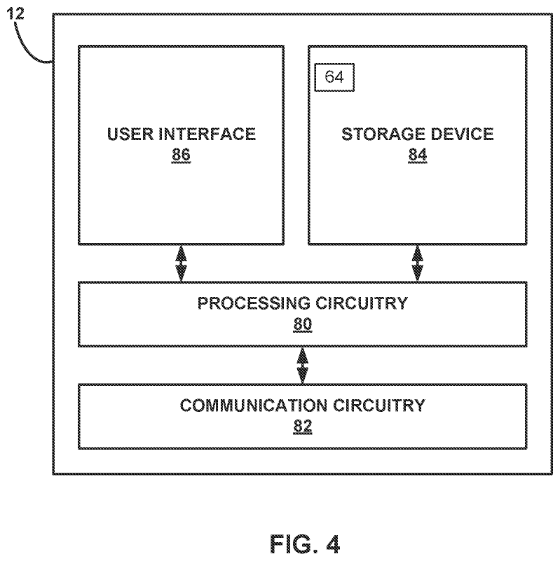

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC, RF communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

External device 12 may transmit data including computer readable instructions which, when implemented by IMD 10, may control IMD 10 to change one or more operational parameters/device settings and/or export collected data. For example, processing circuitry 80 may transmit an instruction to IMD 10 which requests IMD 10 to export collected data (e.g., patient data) to external device 12. In turn, external device 12 may receive the collected data from IMD 10 and store the collected data in storage device 84. The data external device 12 receives from IMD 10 may include various patient data such as cardiac EGM data, which as described herein refers to patient 4's cardiac activity as recorded by IMD 10 in the form of cardiac EGMs. Processing circuitry 80 may implement any of the techniques described herein to analyze the cardiac activity data (and possibly, other patient data) from IMD 10 for indicia of atrial arrythmia(s); for example, by (first) modifying the cardiac activity data to reduce non-atrial activity (e.g., QRS section).

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., evidence levels of heart beat intervals, indications of a detection of an atrial arrythmia, indications of changes in patient health that correlate to the atrial arrythmia detections, and visualizations of various cardiac activity data such as a cardiac EGM. In addition, user interface 86 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

Similar to IMD 10, processing circuitry 80 of external device 12 may be configured with logic 64 that, when executed and run as a computing application, is operative to analyze cardiac activity data of patient 4 for evidence of cardiac episodes. To detect occurrences of cardiac episodes (particularly, atrial arrythmias), the innovative detection method/mechanism (e.g., a machine learning model including a neural network) is programmed into logic 64 such that any application of that method/mechanism is more likely to produce an accurate classification.

FIG. 5 is a block diagram illustrating an example system that includes an access point 90, a network 92, external computing devices, such as a server 94, and one or more other computing devices 100A-100N (collectively, “computing devices 100”), which may be coupled to IMD 10 and local device 90 via network 92, in accordance with one or more techniques described herein. In this example, IMD 10 may use communication circuitry 54 to communicate with local device 90 via a wireless connection. In the example of FIG. 5, local device 90, external device 12, server 94, and computing devices 100 are interconnected and may communicate with each other through network 92.

Local device 90 may be external device 12, in some examples. Local device 90 may include a device that connects to network 92 via any of a variety of connections, such as telephone dial-up, digital subscriber line (DSL), or cable modem connections. In other examples, local device 90 may be coupled to network 92 through different forms of connections, including wired or wireless connections. In some examples, local device 90 may be a user device, such as a tablet or smartphone, that may be co-located with the patient. IMD 10 may be configured to transmit data, such as patient cardiac EGM data and indications of episode data, and/or indications of changes in patient health, to local device 90. Local device 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic CareLink®) Network.

In some examples, one or more of computing devices 100 may be a tablet or other smart device located with a clinician, by which the clinician may program, receive alerts from, and/or interrogate IMD 10. For example, the clinician may access patient data and/or indications of patient health collected by IMD 10 through a computing device 100, such as when patient 4 is in between clinician visits, to check on a status of a medical condition. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 94 includes a storage device 96, e.g., to store data retrieved from IMD 10, and processing circuitry 98. Although not illustrated in FIG. S computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98.

Processing circuitry 98 of server 94 and/or the processing circuity of computing devices 100 may implement any of the techniques described herein to analyze cardiac EGM data received from IMD 10, e.g., to determine whether the data represents an episode of an AT or other atrial tachyarrhythmia. For example, as illustrated in FIG. 5, storage device 96 may store logic for classification of cardiac EGM data according to the techniques described herein. Processing circuitry 98 may implement the techniques of this disclosure according to logic 64, e.g., as described in greater detail below.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

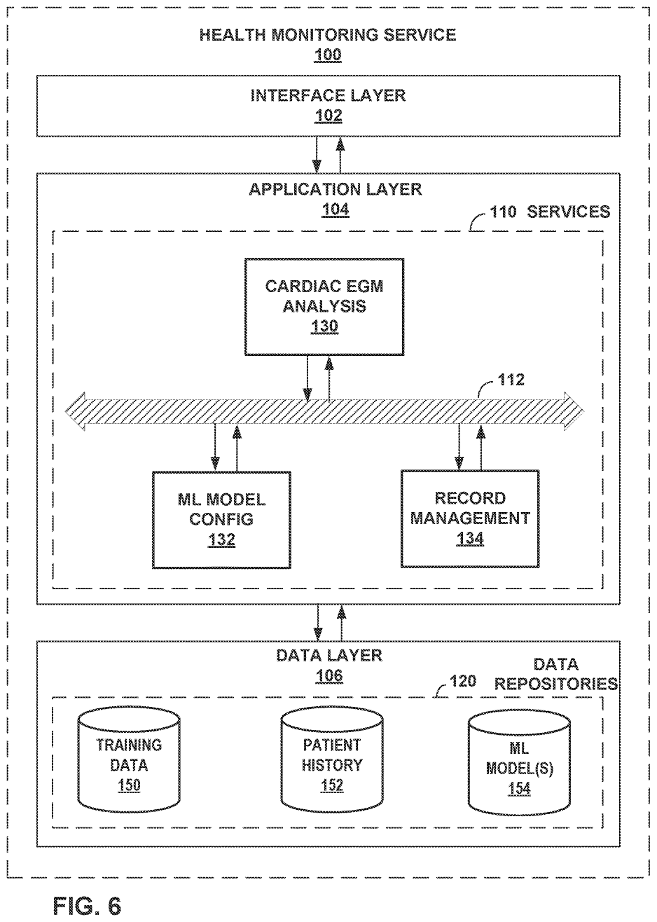

FIG. 6 is a block diagram illustrating a logical perspective of health monitoring service (HMS) 100 operated by the example system of FIG. 5 including functionality to implement HMS 100. An example implementation typically involves various hardware/software components operating on processing circuitry 98 of server 94 and generally configured to be network-accessible for transmitting/receiving various data to/from patient medical devices (e.g., IMD 10), their local devices (e.g., external device 12), computing devices 99, and/or the like.

FIG. 6 provides an operating perspective of HMS 100 when hosted as a cloud-based platform. In the example of FIG. 6, components of HMS 100 are arranged according to multiple logical layers that implement the techniques of this disclosure. Each layer may be implemented by one or more modules comprised of hardware, software, or a combination of hardware and software.

Computing devices, such as external device 12 of FIG. 1, may operate as clients that communicate with HMS 100 via interface layer 102. The computing devices typically execute client software applications, such as desktop application, mobile application, and web applications. Interface layer 102 represents a set of application programming interfaces (API) or protocol interfaces presented and supported by HMS 100 for the client software applications. Interface layer 102 may be implemented with one or more web servers.

As shown in FIG. 6, HMS 102 also includes an application layer 104 that represents a collection of services 110 for implementing the functionality ascribed to HMS herein. Application layer 104 receives information from client applications, e.g., cardiac EGM data for an episode collected by IMD 10 and received from local device 90, and further processes the information according to one or more of the services 104 to respond to the information. Application layer 104 may be implemented as one or more discrete software services 110 executing on one or more application servers, e.g., physical or virtual machines. That is, the application servers provide runtime environments for execution of services 110. In some examples, the functionality of interface layer 102 as described above and the functionality of application layer 104 may be implemented at the same server. Services 110 may communicate via a logical service bus 112. Service bus 112 generally represents a logical interconnection or set of interfaces that allows different services 110 to send messages to other services, such as by a publish/subscription communication model.

Data layer 106 of HMS 100 provides persistence for information in HMS 100 using one or more data repositories 120. A data repository 120, generally, may be any data structure or software that stores and/or manages data. Examples of data repositories 120 include but are not limited to relational databases, multi-dimensional databases, maps, and hash tables, to name only a few examples.

As shown in FIG. 6, each of services 130-134 is implemented in a modular form within HMS 100. Although shown as separate modules for each service, in some examples the functionality of two or more services may be combined into a single module or component. Each of services 130-134 may be implemented in software, hardware, or a combination of hardware and software. Moreover, services 130-134 may be implemented as standalone devices, separate virtual machines or containers, processes, threads or software instructions generally for execution on one or more physical processors.

Cardiac EGM analysis service 130 may be responsive to receipt of cardiac EGM data of an atrial tachyarrhythmia episode from local device 90 indicating that IMD 10 detected an atrial tachyarrhythmia episode (e.g., AT or AF) of patient 4. Cardiac EGM analysis service 130 may initiate performance of any techniques described herein for analyzing and classifying the cardiac EGM data. In some examples, cardiac EGM analysis service 130 may apply a set of features generated from the cardiac EGM data to one or more machine learning models 154 to determine a classification of the cardiac EGM data.

Cardiac EGM analysis service 130 and machine learning models 154 may be an example of logic 64 (FIGS. 3-5). In some examples, instead of or in addition to machine learning models 154 being used by cardiac EGM analysis service 130, they may be used as part of logic 64 by IMD 10 and/or external device 12. The cardiac EGM data and any classification thereof may be stored in patient history 152 by record management service 134.

Machine learning model configuration service 132 may train, validate, and otherwise configure machine learning models 154 using training data 150. A portion of training data 150 may be reserved for validation of machine learning models 154. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Leaming (LWL), Ridge Regression, Least Absolute Shrinkage and Selection Operator (LASSO), Elastic Net, and Least-Angle Regression (LARS), Principal Component Analysis (PCA) and Principal Component Regression (PCR).

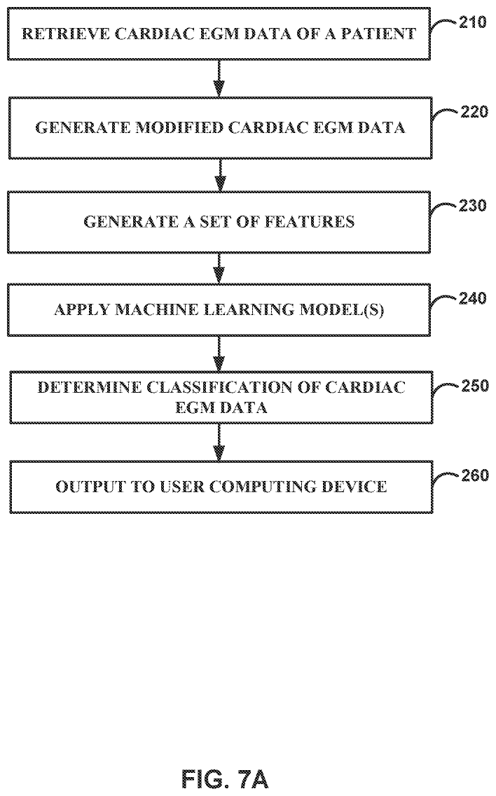

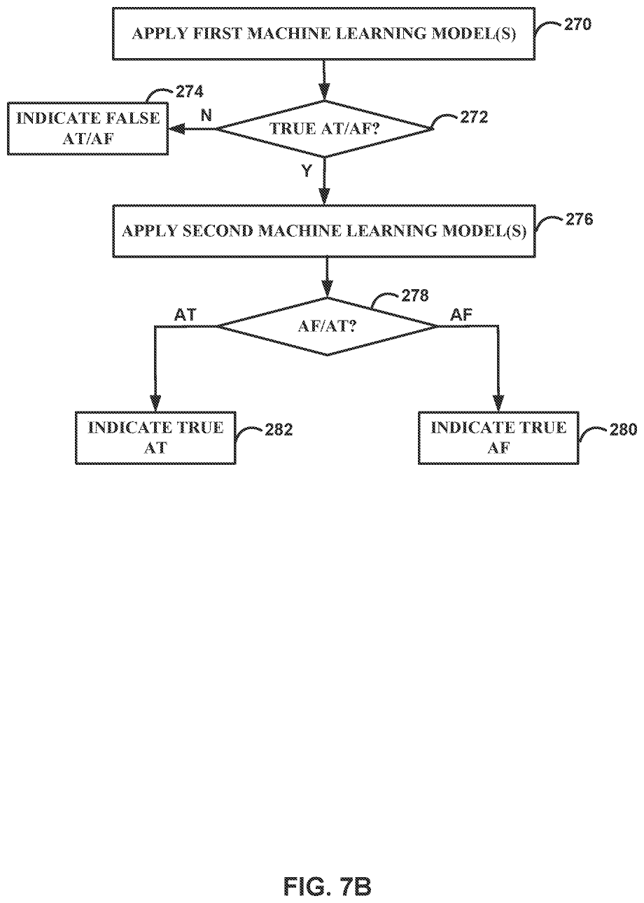

FIG. 7A is a flow diagram illustrating an example operation for detecting an atrial tachyarrhythmia episode, e.g., AT or AF episode, in accordance with one or more examples of the present disclosure. In some examples, the example operation may be implemented, by a medical system including a medical device (e.g., an implantable or insertable medical device) and/or an external device. The following description of the example operation of FIG. 7A uses elements of FIGS. 1-5 to represent any combination of the various embodiments disclosed herein. According to the illustrated example of FIGS. 1-5, processing circuitry 50 of IMD 10, processing circuitry 80 of external device 12, processing circuitry 98 of external device 94, and/or one or more other processors may perform techniques described herein for the example operation.

According to the example of FIG. 7A, processing circuitry of the medical system may retrieve cardiac EGM data of patient 4 (210). In some examples, the cardiac EGM data is a digitized segment of a cardiac EGM signal stored by IMD 10 in response to detecting an atrial tachyarrhythmia, such as AT or AF, using any of the detection techniques described herein. The digitized segment may include a portion of the cardiac EGM that the IMD 10 detected as being the atrial tachyarrhythmia. When the techniques of FIG. 7A are performed by processing circuitry of 50 of IMD 10, retrieving cardiac EGM data may include retrieving the cardiac EGM data from storage device 56. When the techniques of FIG. 7A are performed by processing circuitry of one or more other devices of system 2, such as processing circuitry 98 of server device 94, retrieving cardiac EGM data may include receiving the cardiac EGM data from IMD 10.

In some examples, processing circuitry 50 may monitor cardiac EGM and generate cardiac EGM data continuously, periodically, and/or under certain circumstances. In such examples, processing circuitry 50 of IMD 10 may detect an AT, AF or other atrial tachyarrhythmia episode. This detection by IMD 10 may be an initial atrial arrythmia determination, and IMD may store a cardiac EGM data for a predetermined time period associated with the detection as episode data that may be communicated to external device 12 or server device 94 for adjudication.

In some examples, processing circuitry 98 of server device 94 may receive the cardiac EGM data and perform an adjudication to confirm the initial atrial arrythmia determination by further analyzing such data for indicia of an atrial arrythmia of interest. The adjudication (procedure) may prescribe an analysis of the cardiac activity data in which various types of indicia may be used to determine that the atrial arrythmia of interest has occurred. The atrial arrythmia of interest may refer to an episode of the initial determination (e.g., an AT episode or an AF episode) or a different atrial arrythmia and, possibly, further sub-classification. In the absence of any such indicia, the analysis may be directed towards possible evidence of a false positive by IMD 10.

According to the example technique of FIG. 7A, processing circuitry 98 of server device 94 may generate modified cardiac EGM data based on the retrieved cardiac EGM data (220). Processing circuitry 98 of server device 94 may direct the analysis towards specific cardiac components of the cardiac EGM data over another activity, as explained further below. For example, processing circuitry 98 may modify (at least part) of the cardiac EGM data to reduce QRS complexes in the data, ultimately generating modified cardiac EGM data. Given that atrial activity data provides suitable indicia for any atrial arrythmia of interest, processing circuitry 98 of external device 94 may focus the analysis on atrial activity portions of the cardiac EGM data. In general, the cardiac EGM data described herein may be partitioned a number of ways, for example, into atrial activity and non-atrial activity, which may be further defined by waveform composition.

Processing circuitry 98 may employ a number of techniques to modify the cardiac EGM data. In some examples, processing circuitry 98 identifies QRS portions of the data, e.g., based on its own analysis or markers provided by IMD 10, and replaces segments of the cardiac EGM data including the QRS portions with other data, e.g., a baseline value, or an average or other value determined from QRS or non-QRS portions of the cardiac EGM data. In some examples, processing circuitry 98 may use template matching of an ensemble average template, scaling the template using cross-correlation, and subtraction from the cardiac EGM data. In some examples, processing circuitry 98 may use principal component analysis (PCA) to separate different components of the cardiac EGM data, and then subtract QRS components from the cardiac EGM data. In some examples, processing circuitry 98 applies adaptive filtering techniques to remove QRS complexes from the cardiac EGM data.

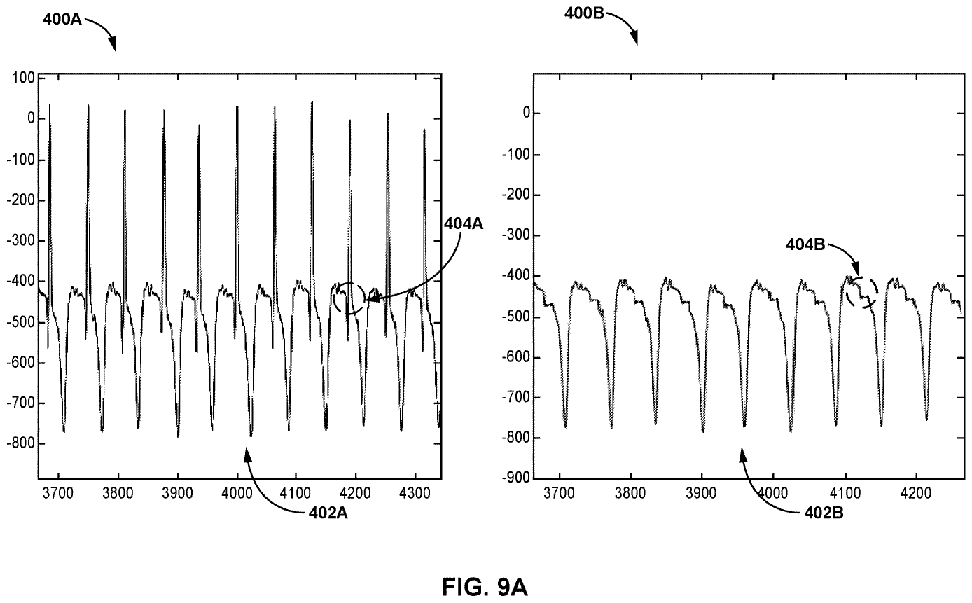

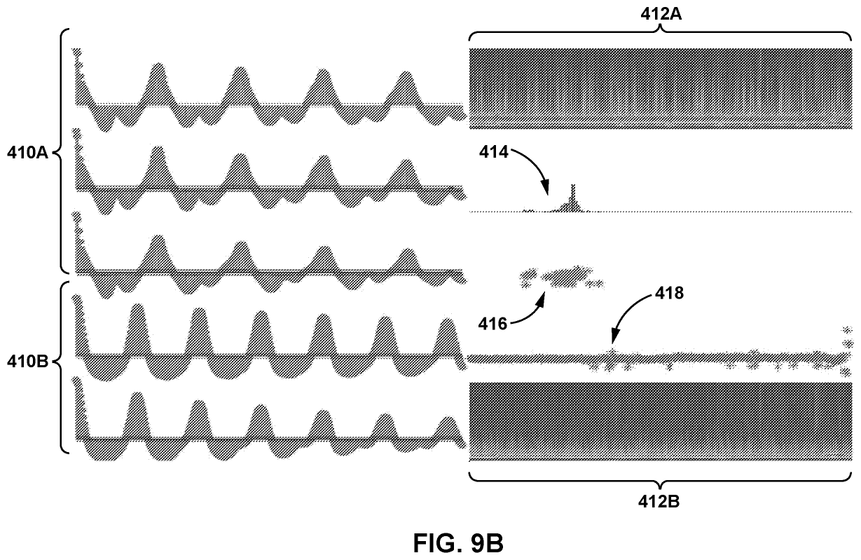

Processing circuitry 98 may generate a set of features (230). Some of the features may be generated from the modified cardiac EGM data. Some of the features may be generated from the unmodified cardiac EGM data. Various example features and techniques for generating the features are discussed below, e.g., with respect to FIGS. 9A and 9B.

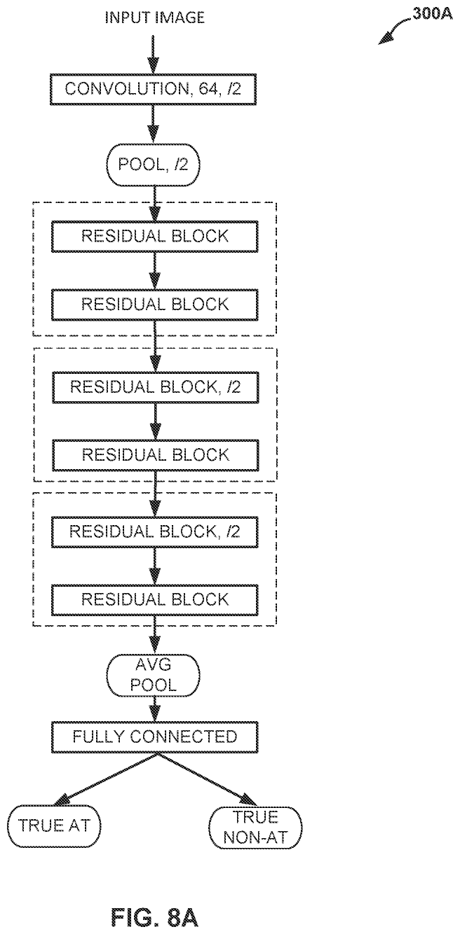

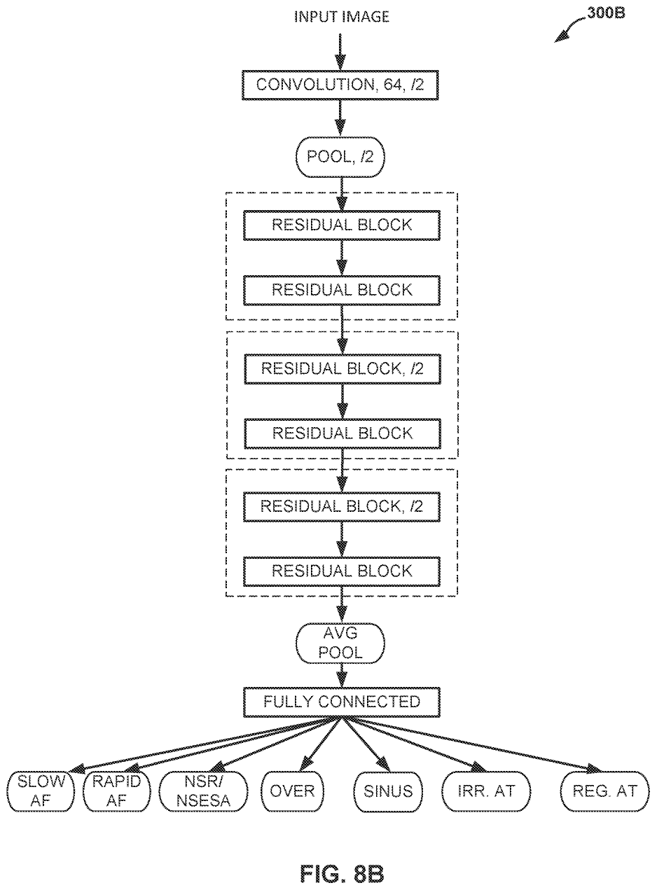

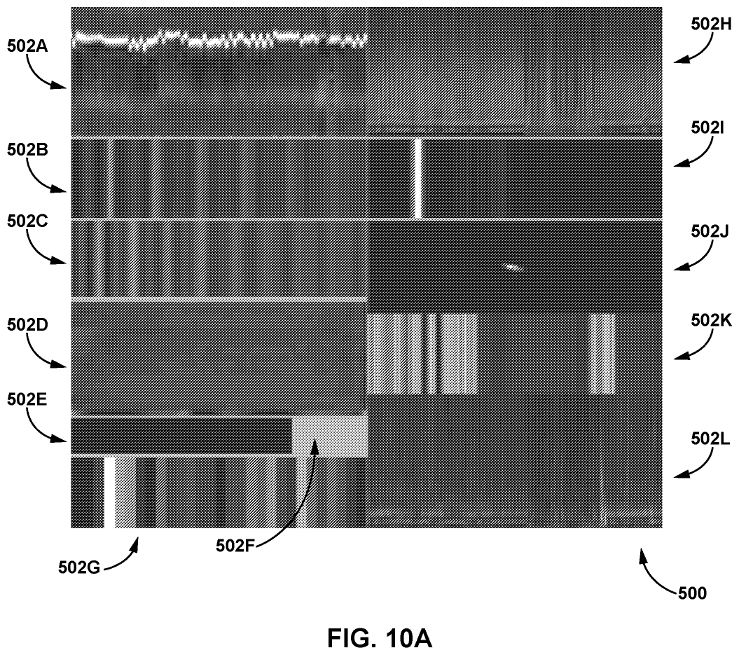

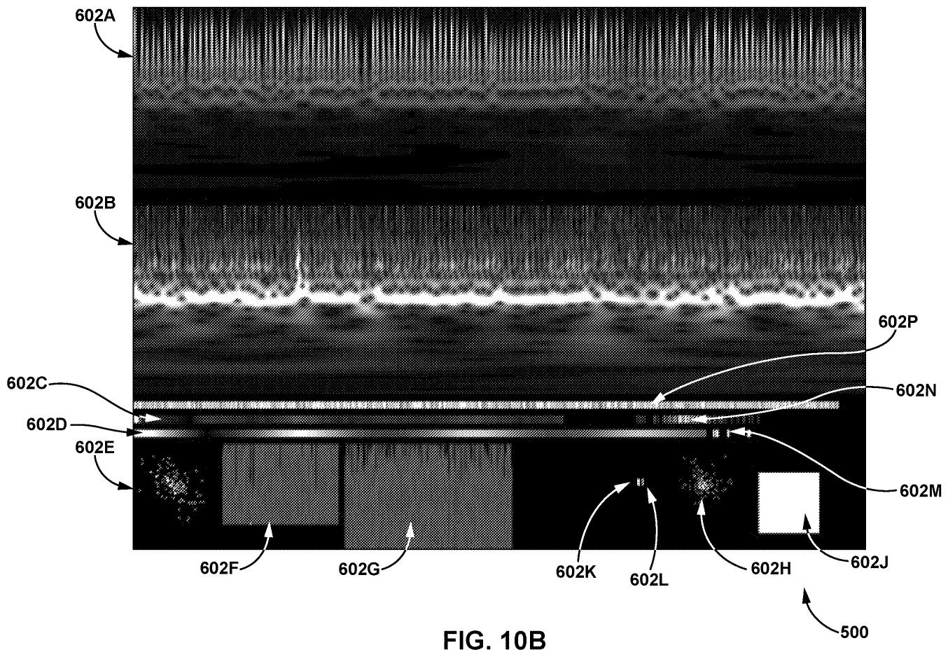

Processing circuitry 98 may apply one or more machine learning models to input data (e.g., input image or other two-dimensional input matrix) comprising the generated features (240), and determine a classification of the cardiac EGM data based on an output of the one or more machine learning models (250). Examples of machine learning models are described with respect to FIGS. 8A and 8B, and an example input matrix is described with respect to FIGS. 10A and 10B. Processing circuitry 98 may output the classification to a user computing device, e.g., computing device 100 (260).

FIG. 7B is a flow diagram illustrating another example operation for determining a classification of an atrial tachyarrhythmia episode, in accordance with one or more examples of the present disclosure. The example operation of FIG. 7B may be an example of application of one or more machine learning models (240) and determining a classification of cardiac EGM data (250) described in FIG. 7A.

In some examples, processing circuitry may apply a machine learning model to the cardiac EGM data for an episode detected by IMD 10 to classify the episode as one of AF, AT, or FALSE, whether the IMD detected the episode as AF or AT. In some examples, as illustrated by FIG. 7B for episodes detected by IMD 10 as AF, the processing circuitry may apply a first one or more machine learning models (270) to the cardiac EGM data, and classify the episode as one of TRUE AF or FALSE AF based on an output of the one or more machine learning models (272). Based on classifying cardiac EGM data as a FALSE AF (N of 272), the processing circuitry may indicate FALSE AF (274). Based on classifying the cardiac EGM data as TRUE AF (Y of 272), the processing circuitry may apply a second one or more machine learning models to the cardiac EGM data (276), to further classify the episode as TRUE AF or TRUE AT (278). Based on the classification (AF or AT of 278), the processing circuitry may indicate TRUE AF (280) or indicate TRUE AT (282).

Similarly, for episodes detected by IMD 10 as AT, the processing circuitry may apply a first machine learning model to classify the episode as one of TRUE AT or FALSE AT, and then apply a second machine learning model to the cardiac EGM data classified as TRUE AT to further classify the episode as AF or AT. The second machine learning model applied to distinguish between AF and AT for true episodes may be the same for both TRUE AT and TRUE AF episodes. In some examples, in order to determine whether AF detection criteria are satisfied, processing circuitry 98 may apply one or more machine learning models, e.g., as described in commonly-assigned U.S. Patent Application Ser. No. 17/329,913, titled “CARDIAC EPISODE CLASSIFICATION,” which is incorporated herein by reference in its entirety. In some examples, one or more machine learning models may be arranged in various stages or other manners to classify cardiac EGM data for an episode detected by IMD 10, e.g., as one of a variety of classifications, as described in greater detail below.

In some examples, different input features, e.g., images or other two-dimensional arrays, and machine learning models may be utilized by processing circuitry 98 to determine different classifications and subclassifications, e.g., in a staged manner. In some examples, a respective set of one or more features generated from the ECG data or modified ECG data may be input into a respective one of a plurality of machine learning models, e.g., neural networks, which may be combined using an ensemble, e.g., ensemble neural network. In some examples, a single set of features, e.g., arranged as an input array, may be provided as input to a machine learning model architecture that provides outputs, e.g., probabilities, for any number of possible classifications. Example classifications include: atrial tachycardia or non-atrial tachycardia; atrial fibrillation or non-atrial fibrillation; irregular atrial tachycardia, irregular non-atrial tachycardia, regular tachycardia, regular non-atrial tachycardia, or atrial fibrillation; irregular atrial tachycardia, normal sinus rhythm, oversensing, sinus tachycardia, slow atrial fibrillation, or rapid atrial fibrillation; irregular atrial tachycardia, normal sinus rhythm, oversensing, sinus tachycardia, slow atrial fibrillation, rapid atrial fibrillation, or regular atrial tachycardia; or any combination of any of these classifications.

Furthermore, although described herein generally in the context of examples in which IMD 10 detects atrial tachyarrhythmias using non-machine learning techniques, and stores episode data for further analysis by another computing device or system using the techniques described herein, the techniques are not limited to such examples. In some examples, IMD 10 may implement one or more machine learning models to identify or initially classify atrial tachyarrhythmias. The analysis of cardiac EGM data may be according to the techniques described herein, or other machine learning techniques. The models employed by IMD 10 may be less complex or processing intensive than those employed by other computing devices or systems. In some examples, IMD 10 may transmit the episode data to another computing device or system, e.g., external device 12 or server device 94, for classification/confirmation using the techniques described herein.

Additionally, the techniques described herein may selectively be applied or not applied to cardiac EGM data of episodes detected by IMD 10 based on one or more criteria. In some examples, processing circuitry, e.g., processing circuitry 98, bypasses the techniques of this disclosure, e.g., allowing the episode type detected by IMD 10 to be reported to a clinician or other user. Example criteria for bypassing the techniques of this disclosure, e.g., including application of one or more machine learning models to a set of features generated from EGM data and modified EGM data, include duration of the episode, heart rate during the episode, or whether the episode was associated with an indication that the patient experienced symptoms.

FIGS. 8A and 8B illustrate example machine learning models, in accordance with one or more examples of the present disclosure. Both FIG. 8A and FIG. 8B are illustrations of a machine learning model architecture comprised of at least one residual block, Residual blocks in a residual architecture, e.g., for a convolutional neural network (CNN), may have convolution layers, one or more parallel signal feedthrough layers, and one or more pooling layers.

As illustrated in FIGS. 8A and 8B, each of machine learning models 300A and 300B includes a convolution layer that received an input image, a pooling layer, a number of residual blocks, and average pooling layer, and a fully connected layer that provides respective probabilities for output classes usable by processing circuitry to determine a classification of the cardiac EGM data used to generate the input image. Machine learning model 300A includes two output classes: True AT and True non-AT. Machine learning model 300B includes seven output classes: Slow AF, Rapid AF, normal sinus rhythm (e.g., with ectopy), oversensing, sinus tachycardia, irregular AT, and regular AT.

Residual connections are a popular element in CNN architectures. Using residual connections improves gradient flow through the network and enables training of deeper networks. Residual connections may enable a more complex graph structure in which layers can have inputs from multiple layers and outputs to multiple layers, as opposed to a neural network that consists of a single sequence of layers.

These types of networks are often called directed acyclic graph (DAG) networks. A residual network (ResNet) is a type of DAG network that has residual (or shortcut) connections that bypass the main network layers. Residual connections enable the parameter gradients to propagate more easily from the output layer to the earlier layers of the network, which makes it possible to train deeper networks. This increased network depth can result in higher accuracies on more difficult tasks.

FIG. 9A and B depict graphical representations of cardiac EGM data sensed by IMD 10 of FIGS. 1-3, and features derived from the cardiac EGM data. FIGS. 9A and 9B illustrate techniques for generating a set of features from the cardiac EGM data for application to a machine learning model to determine a classification of the cardiac EGM data.

FIG. 9A includes a graph 400A of raw cardiac EGM data 402A and a graph 400B of modified cardiac EGM data 402B. Modified cardiac EGM data 402B has been modified by processing circuitry, e.g., processing circuitry 98, to reduce one or more QRS complexes, e.g., sections including QRS complexes, in the cardiac EGM data. In some examples, the processing circuitry applies a filter to cardiac EGM data 402A to produce modified cardiac EGM data 402B. The filter may be applied to sections 404A of cardiac EGM data 402A including QRS complexes to produce modified sections 404B. The filter may decrease at least one amplitude value in section 404B relative to the value in section 404A. In some examples, the filter is applied as a moving filter. In some examples, the filter applied is an adaptive filter.

In some examples, the processing circuitry determines sections 404A and 404B by setting a window around a detected R-wave, e.g., R-wave peak. In some examples, the processing circuitry replaces each sample amplitude value within the window with an average of at two other values within the window, e.g., an average of a first sample value and a last sample value within the window, or an average of other preceding/following sample values. In some examples, all values within the window are replaced with an average of two or more selected values. In some examples, the processing circuitry replaces segments of the cardiac EGM data including the QRS portions with an average or other value determined from QRS or non-QRS portions of the cardiac EGM data. In some examples, processing circuitry may use template matching of an ensemble average template, scaling the template using cross-correlation, and subtraction from the cardiac EGM data to diminish the QRS segments. In some examples, processing circuitry 98 may use principal component analysis (PCA) to separate different components of the cardiac EGM data, and then subtract QRS components from the cardiac EGM data.

FIG. 9B graphically represents example features that are generated from the raw cardiac EGM data 402A and/or the modified cardiac EGM data 402B. For example, the processing circuitry, e.g., processing circuitry 98 of server device 94, may generate at least one of an autocorrelogram 410A of cardiac EGM data 402A or an autocorrelogram 410B of modified cardiac EGM data 402B. To generate each of autocorrelograms 410, the processing circuitry may determine, for each sample of data 402, a metric of similarity or correlation between the sample and a lagged sample (e.g., preceding by the sample by N samples) from data 402A,402B. The processing circuitry may plot the correlation/similarity metrics as an autocorrelogram.

As another example, the processing circuitry, e.g., processing circuitry 98 of server device 94, may generate at least one of a scalogram 412A of cardiac EGM data 402A or a scalogram 412B of modified cardiac EGM data 402B as features of a set of features to be input into a machine learning model. To generate a scalogram, the processing circuitry may transform the data 402, e.g., apply a wavelet transform to the data. In some examples, transformation of the data may include applying an Analytic Morelet wavelet or other continuation wavelet transform (CWT) to the data.

In some examples, features of the set of features derived from the cardiac EGM data may additionally and/or alternatively include one or more of wavelet scattering of raw cardiac EGM data 402A, wavelet scattering of QRS diminished EGM data, or episode duration, such as duration of the episode that resulted in the EGM data.

In some examples, the processing circuitry generates at least one feature of the set of features by determining RR intervals of cardiac EGM data 402A, and plotting the determined RR intervals. For example, the processing circuitry may plot of histogram 414 or Lorenz plot 416 of RR intervals within cardiac EGM data 402A. In some examples, processing circuitry may additionally or alternatively generate a scaled version of the Lorenz plot. In some examples, the processing circuitry may plot a scatter plot 418 of RR intervals. In some examples, the processing circuitry may identify RR intervals in the data exceeding a threshold length, and plot those intervals.

Long RR intervals may be indicative of slow Atrial Flutter (AFL) episodes. In some examples, the processing circuitry may extract a segment of the data corresponding to an RR interval greater than the threshold, e.g., 750 milliseconds, and include the extracted segment as a feature in the set of features. In some examples, the extracted segments may be P-wave segments, e.g., a segment of the cardiac EGM data centered on a sample preceding an R-wave of an identified RR interval and within which atrial activity would be expected to occur.

Another example feature is based on a count of flutter waves in the cardiac EGM data. The samples in the feature may represent one or more of a number of flutter waves in the cardiac EGM data, a number of P-waves in the cardiac EGM data, or a score based on satisfaction of one more criteria by the number of flutter wave and/or the number of P-waves, e.g., flutter waves exceeding a threshold and/or P-wave being less than a threshold.

As another example, processing circuitry generates a feature based on a score representing the evidence of AT in the cardiac EGM data calculated based on one or more measures of regularity and/or irregularity of RR intervals in cardiac EGM data. Example measures of regularity and/or irregularity of RR intervals include modesum, median, interquartile range, amount (number or percentage) shorter than a threshold, or sudden shortening in sequential RR intervals. Example measures and techniques for determining such measures are described in commonly-assigned U.S. patent application Ser. No. 17/339,308, Titled “detection of Atrial Tachycardia Based on REGULARITY OF CARDIAC RHYTHM,” and incorporated herein by reference in its entirety.

As another example, processing circuitry generates a feature based on a history of atrial tachyarrhythmia (e.g., AT and/or AF) prior to the episode that resulted in the EGM data. For example, processing circuitry may generate a feature based on amounts of time the patient experienced atrial tachyarrhythmia (e.g., AT and/or AF) each day for a number of days, e.g., 30 days, prior to the episode, e.g., 206 mins on 30^{th} day, 265 mins on 29^{th} day, 100 mins on 28^{th} day, and so on. As another example, processing circuitry generates a feature based on a duration of the episode that resulted in the EGM data.

FIG. 10A illustrates an example two-dimensional array 500 of samples that can be input into the example machine learning models to classify cardiac EGM data from an atrial tachyarrhythmia episode according to the techniques described herein. In the illustrated example, two-dimensional array 500 is an image, e.g., a grayscale image. Processing circuitry, e.g., processing circuitry 98 of server device 94, may construct array 500 in a two-dimensional matrix form to include each of a set of features as described herein. The machine learning model may include a neural network architecture, such as the examples depicted in FIGS. 8A and 8B, with an initial layer configured to accept two-dimensional array 500 as input.

As illustrated in FIG. 10A, array 500 may include a plurality of sub-arrays 502A-502L (collectively, “sub-arrays 502”). Each of sub-arrays 502 may correspond to a respective features of a set of features. In the example illustrated by FIG. 10A, sub-arrays 502 are sub-images. To generate each of sub-arrays 502, the processing circuitry may determine the corresponding feature, and normalize (e.g., to a 0 to 255 scale) and convert the feature to a grayscale image. The number of types of sub-arrays 502 and corresponding features depicted in FIG. 10A is one example. In other examples, more or fewer sub-arrays 502 and features may be included in an array 500.

In an example implementation where the machine learning model includes a residual neural network, input array 500 may be increased in size to incorporate multiple features. The residual neural network may be designed to accommodate any array size. Other examples of input array 500 may combine even more feature sub-arrays without substantially increasing processing time or complexity.

In some examples, the processing circuitry may generate array 500 in a way that applies different weights to respective features of the set of features in the analysis using machine learning model(s) 154. In particular, a relative size and location of a sub-array 502 within array 500 may corelate to its influence on the output of machine learning model(s) 154. Consequently, different features of the set of features may be given different weights based on the relative size and location of the corresponding sub-arrays 502 in array 500.

In the example illustrated in FIG. 10A, sub-array 502A comprises one or more segments of cardiac EGM data 402A associated with P-waves. To generate sub-array 502A, the processing circuitry may identify R-waves, extract segments centered on a sample preceding the R-wave, and create a sample array of the extracted segments. The sample array may then be normalized and converted to grayscale.

Sub-array 502B is an autocorrelogram 410A of cardiac EGM data 402A, and sub-array 502C is an autocorrelogram 410B of modified cardiac EGM data 402B. Sub-array 502D includes one or more P-wave segments extracted with RR intervals that exceed a threshold length. Sub-array 502E is a greyscale plot of a feature based on a count of flutter waves in the cardiac EGM data, e.g., a plot of one or more of a number of flutter waves in the cardiac EGM data, a number of P-waves in the cardiac EGM data, or a score based on satisfaction of one more criteria by the number of flutter wave and/or the number of P-waves. Sub-array 502F is a grayscale plot of a score representing the evidence of AT in the cardiac EGM data calculated based on one or more measures of regularity and/or irregularity of RR intervals in cardiac EGM data. Sub-array 502G is a grayscale plot representing, for each day of 30 days preceding the episode for which the cardiac EGM data was stored, a number of minutes the patient was within AT or AF, with the numbers normalized to a 0 to 255 range.

Sub-array 502H is a scalogram 412A of cardiac EGM data 402A, and sub-array 502L is a scalogram 412B of modified cardiac EGM data 402B. Sub-array 502I is a histogram plot 414 and sub-array 502J is a Lorenz plot 416 of RR intervals in cardiac EGM data 402A. Sub-array 502K is a scatter plot 418 of RR intervals in cardiac EGM data 402A.

In some examples, the cardiac EGM data may be segmented, and processing circuitry may determine different features and sub-arrays for each segment. For example, processing circuitry may determine respective autocorrelograms or scalograms of each segment for inclusion in an array as separate sub-arrays.

FIG. 10B illustrates an example two-dimensional array 600 of samples that can be input into the example machine learning models to classify cardiac EGM data from an atrial tachyarrhythmia episode according to the techniques described herein. In the illustrated example, two-dimensional array 600 is an image, e.g., a grayscale image. Processing circuitry, e.g., processing circuitry 98 of server device 94, may construct array 600 in a two-dimensional matrix form to include each of a set of features as described herein. The machine learning model may include a neural network architecture, such as the examples depicted in FIGS. SA and 8B, with an initial layer configured to accept two-dimensional array 600 as input.

As illustrated in FIG. 10A, array 600 may include a plurality of sub-arrays 602A-602P (collectively, “sub-arrays 602”). Each of sub-arrays 602 may correspond to a respective features of a set of features. In the example illustrated by FIG. 10B, sub-arrays 602 are sub-images. To generate each of sub-arrays 602, the processing circuitry may determine the corresponding feature, and normalize (e.g., to a 0 to 255 scale) and convert the feature to a grayscale image. The number of types of sub-arrays 602 and corresponding features depicted in FIG. 10B is one example. In other examples, more or fewer sub-arrays 602 and features may be included in an array 600.

In an example implementation where the machine learning model includes a residual neural network, input array 600 may be increased in size to incorporate multiple features. The residual neural network may be designed to accommodate any array size. Other examples of input array 600 may combine even more feature sub-arrays without substantially increasing processing time or complexity.

In some examples, the processing circuitry may generate array 600 in a way that applies different weights to respective features of the set of features in the analysis using machine learning model(s) 154. In particular, a relative size and location of a sub-array 602 within array 600 may corelate to its influence on the output of machine learning model(s) 154. Consequently, different features of the set of features may be given different weights based on the relative size and location of the corresponding sub-arrays 602 in array 600.

In the example illustrated in FIG. 10B, sub-array 602A is a scalogram of raw cardiac EGM data 402A, and sub-array 602B is a scalogram of QRS diminished cardiac EGM data 402B. Sub-array 602C is an autocorrelogram of raw cardiac EGM data 402A. and sub-array 602D is an autocorrelogram 410B of QRS diminished cardiac EGM data 402B. Sub-array 602E is a Lorenz plot of raw cardiac EGM data 402A using difference of RR intervals. Sub-array 602H is a Lorenz plot of raw cardiac EGM data 402A using RR intervals. Sub-array 602F is an amplitude plot of raw cardiac EGM data 402A. Sub-array 602G is a plot of long RR intervals, such as greater than 750 ms, in raw cardiac EGM data 402A. Sub-array 602J is a plot corresponding to episode duration of raw cardiac EGM data 402A. For example, the longer the episode duration of the raw cardiac EGM data 402A, the larger the box corresponding to sub-array 602J will be. Sub-array 602K is a grayscale plot of a score representing the AT evidence in the cardiac EGM data calculated based on one or more measures of regularity and/or irregularity of RR intervals in cardiac EGM data. Sub-array 602L is a grayscale plot of a feature based on a count of flutter waves in the cardiac EGM data, e.g., a plot of one or more of a number of flutter waves in the cardiac EGM data, a number of P-waves in the cardiac EGM data, or a score based on satisfaction of one more criteria by the number of flutter wave and/or the number of P-waves. Sub-array 602M is a grayscale plot representing an AT or AF trend of cardiac EGM data. Sub-array 602N is a histogram plot of RR intervals in raw cardiac EGM data 402A. Sub-array 602P is a scatter plot of RR intervals in raw cardiac EGM data 402A.

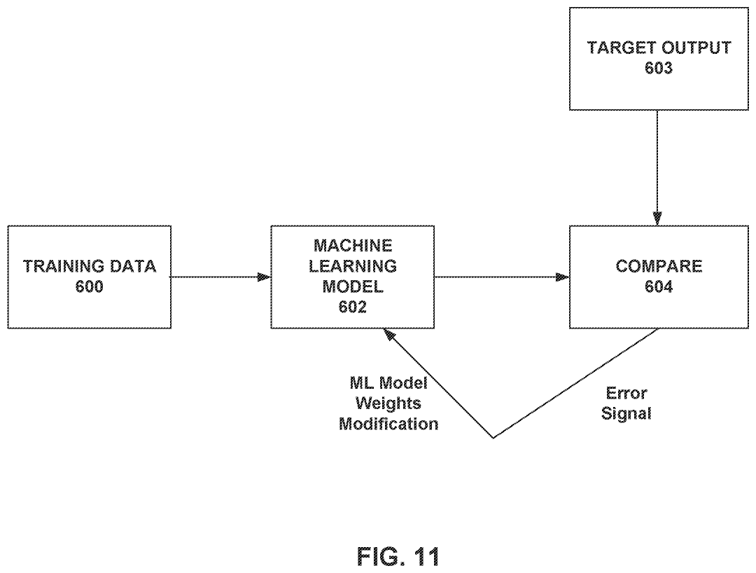

FIG. 11 is an example of a machine learning model 602, which may be an example of machine learning models 154, 300A, and 300B, being trained using supervised and/or reinforcement learning techniques. The machine learning model 602 may be implemented using any number of models for supervised and/or reinforcement learning, such as but not limited to, an artificial neural network, a decision tree, naïve Bayes network, support vector machine, or k-nearest neighbor model, to name only a few examples. In some examples, one or more of IMD 10, external device 12, server 94, and/or computing device(s) 99 initially trains the machine learning model 602 based on a training sets of features corresponding to different arrhythmia classifications. In some examples, one or more machine learning models 602 may be developed by machine learning model configuration service 132 (FIG. 6) by identifying training data 600, which may be an example of training data 150 (FIG. 6) in cardiac EGM data from IMDs 10 for episodes of AT and AF.

Training data 600 may include a set of feature vectors, where each feature in the feature vector represents a value for a particular metric. One or more of IMD 10, external device 12, server device 94, and/or computing device(s) 99 may select a training set comprising a set of training instances, each training instance comprising an association between one or more respective cardiac EGM features and a respective arrhythmia classification. One or more experts may annotate the cardiac EGM data with one or more target classifications, e.g., the classifications discussed herein, and machine learning model configuration service 132 (FIG. 6) may generate sets of features and images 500 (containing sub-images for the respective features) labeled with the annotation to create training data 600.

A prediction or classification by the machine learning model 602 may be compared 604 to the target output 603, e.g., which may be based on the labeled classification, and an error signal and/or machine learning model weights modification may sent/applied to the machine learning model 602 based on the comparison to modify/update the machine learning model 602. For example, one or more of IMD 10, external device 12, server 94, and/or computing device(s) 99 may, for each training instance in the training set, modify, based on the respective cardiac EGM features and the respective classification of the training instance, the machine learning model 602 to change a score generated by the machine learning model 602 in response to subsequent sets of features applied to the machine learning model 602. As discussed herein, the features from the cardiac EGM data and modified cardiac EGM data included in feature sets according to the techniques of this disclosure were selected by experts based on their knowledge of atrial arrhythmia and corresponding cardiac EGM features. A portion of training data 600 may be reserved for model validation.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1. A medical system comprising:
a medical device configured to store cardiac electrogram data of a patient from a predetermined time period associated with an episode of atrial tachyarrhythmia of the patient detected by the medical device; and
processing circuitry configured to:
generate a set of features from the cardiac electrogram data, wherein, to generate at least one feature of the set of features, the processing circuitry is configured to modify the cardiac electrogram data to reduce QRS complexes in the cardiac electrogram data;
determine a classification of the cardiac electrogram data from a plurality of predetermined classifications based on application of a machine learning model to the set of features, the plurality of classifications including at least one atrial tachycardia classification; and
output an indication of the determined classification for presentation to a user via a computing device.

2. The medical system of claim 1, wherein the plurality of classifications comprises atrial tachycardia and non-atrial tachycardia.

3. The medical system of claim 1, wherein the plurality of classifications comprises atrial fibrillation and non-atrial fibrillation.

4. The medical system of claim 1, wherein the plurality of classifications comprises irregular atrial tachycardia, irregular non-atrial tachycardia, regular atrial tachycardia, regular non-atrial tachycardia, and atrial fibrillation, or
the plurality of classifications comprises irregular atrial tachycardia, normal sinus rhythm, oversensing, sinus tachycardia, slow atrial fibrillation, and rapid atrial fibrillation.

5. The medical system of claim 1, the processing circuitry is configured to determine the classification based on respective probabilities of output classes generated by the machine learning model.

6. The medical system of claim 1, wherein to modify the cardiac electrogram data to reduce QRS complexes in the cardiac electrogram data, the processing circuitry is configured to apply a filter to the cardiac electrogram data, wherein the filter is configured to decrease at least one amplitude value of QRS sections of the cardiac electrogram data.

7. The medical system of claim 6, wherein the filter comprises a moving filter and the processing circuitry is further configured to, for every R-peak sample, replace each sample value within a window with an average of at least two sample values within the window.

8. The medical system of claim 1, wherein the set of features comprises at least one of a scalogram of the cardiac electrogram data or a scalogram of the modified cardiac electrogram data.

9. The medical system of claim 1, wherein the set of features comprises at least one of an autocorrelogram of the cardiac electrogram data or an autocorrelogram of the modified cardiac electrogram data.

10. The medical system of claim 1, wherein to generate at least one feature of the set of features, the processing circuitry is configured to:
determine RR intervals of the cardiac electrogram data; and
plot the determined RR intervals.

11. The medical system of claim 1, wherein to generate at least one feature of the set of features, the processing circuitry is configured to:
identify one or more RR intervals in the cardiac electrogram data exceeding a threshold length; and
for each of the one or more identified RR intervals, extract a P-wave segment from the cardiac electrogram data.

12. The medical system of claim 1, wherein to generate at least one feature of the set of features, the processing circuitry is configured to:
identify flutter waves within the cardiac electrogram data; and
determine a count of the identified flutter waves.

13. The medical system of claim 1, wherein to generate at least one feature of the set of features, the processing circuitry is configured to:
extract a plurality of P-wave segments from the cardiac electrogram data; and
plot the extracted P-wave segments.

14. The medical system of claim 1, wherein to determine the classification, the processing circuitry is configured to:
generate a two-dimensional array of samples comprising an arrangement of the set of features as respective sub-arrays of the two-dimensional array; and
apply the two-dimensional array of samples as input to the machine learning model.

15. The medical system of claim 14, wherein the processing circuitry is configured to apply a respective weight to each feature of the set of features by determining a size of the respective sub-array in the two-dimensional array.

16. The medical system of claim 1, wherein the two-dimensional array of samples is an image and the plurality of sub-arrays are sub-images within the image.

17. The medical device system of claim 1, wherein the medical device comprises an insertable cardiac monitor comprising:
a hermetically sealed housing configured for subcutaneous implantation within the patient,
wherein the housing has a length from a first end to a second end, a width, and a depth,
wherein the length is greater than the width and the width is greater than the depth,
wherein the length is within a range from 5 millimeters (mm) to 60 mm,
wherein the width is within a range from 5 mm to 15 mm, and
wherein the depth is within a range from 5 mm to 15 mm;
the processing circuitry within the housing;
a power source within the housing and operatively coupled to the processing circuitry;
a memory within the housing and operatively coupled to the processing circuitry;
sensing circuitry within the housing and operatively coupled to the processing circuitry;
a first electrode at or proximate the first end of the housing and operatively coupled to the sensing circuitry; and
a second electrode at or proximate the second end of the housing and operatively coupled to the sensing circuitry.

18. A non-transitory computer-readable storage medium comprising program instructions that, when executed by processing circuitry of a medical system, cause the processing circuitry to perform:
generating a set of features from cardiac electrogram data of a patient, wherein generating at least one feature of the set of features comprises modifying the cardiac electrogram data to reduce QRS complexes in the cardiac electrogram data, wherein the cardiac electrogram data was stored by a medical device and is from a predetermined time period associated with an episode of atrial tachyarrhythmia of the patient detected by a medical device;
determining a classification of the cardiac electrogram data from a plurality of predetermined classifications based on application of a machine learning model to the set of features, the plurality of classifications including at least one atrial tachycardia classification; and
outputting an indication of the determined classification for presentation to a user via a computing device.

19. A medical system comprising:
a medical device configured to store cardiac electrogram data of a patient from a predetermined time period associated with an episode of atrial tachyarrhythmia of the patient detected by the medical device; and
means for performing:
generating a set of features from the cardiac electrogram data of the patient, wherein generating at least one feature of the set of features comprises modifying the cardiac electrogram data to reduce QRS complexes in the cardiac electrogram data;
determining a classification of the cardiac electrogram data from a plurality of predetermined classifications based on application of a machine learning model to the set of features, the plurality of classifications including at least one atrial tachycardia classification; and
outputting an indication of the determined classification for presentation to a user via a computing device.

20. The medical system of claim 1, wherein the processing circuitry comprises at least one of processing circuitry of an external computing device in communication with the medical device or a cloud computing system in communication with the medical device.
